(19) United States
(12) Patent Application Publication
(10) Pub. No.: US 2024/0082279 A1
(43) Pub. Date: Mar. 14, 2024

(54) COMPOSITIONS AND METHODS FOR PREVENTING AND/OR TREATING SCARS

(71) Applicant: L'Oreal, Paris (FR)

(72) Inventors: I-Chien LIAO, Princeton, NJ (US); Charbel Bouez, Hoboken, NJ (US); Chunmei Ding, Shanghai (CN); Chunyan He, Shanghai (CN); Qian Zheng, Bridgewater, NJ (US)

(21) Appl. No.: 17/900,094

(22) Filed: Aug. 31, 2022

(51) Int. Cl. A61K 31/7004; A61B 18/20

(52) U.S. Cl. CPC A61K 31/7004; A61B 18/203; A61B 2018/00577

(57) ABSTRACT

The disclosure relates to compositions and methods for preventing, reducing, and/or treating scars. The compositions comprise at least one C-glycoside or derivative thereof, and the methods comprise treating skin and/or scar tissue with at least one C-glycoside or derivative thereof and at least one skin modification stimulus.

### TECHNICAL FIELD

The disclosure relates to compositions and methods for preventing and/or treating scars. The compositions comprise at least one C-glycoside or derivative thereof, and the methods comprise treating skin and/or scar tissue with at least one C-glycoside or derivative thereof and at least one skin modification stimulus.

### BACKGROUND

Scars, which may occur as part of the skin's natural healing process following injury from burns, lacerations, abrasion, acne, skin infection, animal bites, piercings, surgery, etc., form after the wound is healed. Types of scars include, for example, atrophic scars (including acne scars), hypertrophic scars (including raised scars and keloid scars), contractive scars, and stretch mark scars.

Generally a wound site goes through stages of inflammation, granulation, and matrix remodeling during the healing process. The inflammation stage begins shortly after the injury and lasts for several days, and the granulation stage begins about 24 hours after the injury and lasts for one to two weeks. The remodeling stage lasts up to about a year and involves collagen cross-linking and replacement, wherein a new collagen matrix making up the scar tissue forms.

The signaling pathway of transforming growth factor beta-1 (TGF-β1) is of particular importance to scar formation. TGF-β1 has been shown to be present at high levels in post burn hypertrophic scars, and to be over-expressed in fibroblasts isolated from keloid tissue. Thus, scars are believed to be caused, in part, by the signal transduction of TGF-β1 that triggers the conversion of fibroblasts to myofibroblasts, which ultimately results in formation of thick, fibrous scar tissue at the wound site.

Many individuals wish to reduce or improve the appearance of scars, and therefore seek scar treatment. For example, retinoids, topical or injectable steroids, and silicone dressings are currently used to reduce or improve the appearance of scars. Additionally, surgical or non-surgical procedures that remove of parts of the epidermis can accelerate the renewal of the skin's surface, and thus reduce the appearance of scars. For example, clinicians may use scar modification techniques such as laser treatments and chemical peels to reduce a scar's appearance, and to even reduce the formation of scars after surgery. Although such treatments cannot completely remove or prevent scars, it can make them less noticeable and uncomfortable.

The development of improved scar management options is important to improve the quality of life of patients suffering from undesirable scar appearance. Thus, there is an ongoing need for improved skin treatments to prevent the formation of scars, or to treat scars that have already formed.

It has now been surprisingly discovered that C-glycosides and derivatives thereof such as proxylane (also known as Pro-xylane™ and hydroxypropyl tetrahydropyrantriol) have an unexpected advantage in treating scars in that these compounds are particularly effective at minimizing the occurrence and/or appearance of scars, thereby improving the appearance of the skin.

### SUMMARY

The present disclosure relates to compositions and methods for treating scars, for example reducing scar tissue, improving the appearance of a scar, and/or preventing or reducing the formation of scar tissue. Compositions according to the disclosure comprise at least one C-glycoside or derivative thereof, and methods according to the disclosure combine the use of at least one C-glycoside or derivative thereof with treatment of skin or scar tissue using a skin modification stimulus.

In various embodiments, the disclosure relates to the use of at least one C-glycoside or derivative thereof in conjunction with various aesthetic procedures for preventing, reducing, and/or improving the appearance of scars. In some embodiments, the disclosure relates to methods comprising applying at least one C-glycoside or derivative thereof before, during, and/or after a skin surfacing or scar modification treatment.

In various embodiments, the disclosure relates to methods for treating scar tissue, for example by reducing the appearance of the scar, comprising applying at least one C-glycoside or derivative thereof to the scar tissue in combination with treatment with a skin modification stimulus. For example, in some embodiments, the disclosure relates to methods for treating scar tissue, the methods comprising:
(1) treating scar tissue with at least one skin modification stimulus, and
(2) applying at least one C-glycoside or derivative thereof to the scar tissue.

In other embodiments, the disclosure relates to methods for preventing or reducing the formation of scar tissue, comprising applying at least one C-glycoside or derivative thereof to skin that does not have scar tissue prior to a skin injury, e.g. prior to a surgical procedure, in combination with a skin modification stimulus treatment. For example, in some embodiments, the disclosure relates to methods for preventing or reducing the formation of scar tissue, the methods comprising:
(1) treating uninjured skin with at least one skin modification stimulus, and
(2) applying at least one C-glycoside or derivative thereof to the skin.

In another embodiment, the disclosure relates to methods of treating skin to prevent or reduce the formation of scar tissue after surgery, the methods comprising:
(1) injuring skin, and
(2) applying at least one C-glycoside or derivative thereof to the skin.

In various embodiments, the at least one C-glycoside or derivative thereof may be applied to skin or scar tissue during treatment with a skin modification stimulus. In other embodiments, the at least one C-glycoside or derivative thereof may be applied to the skin or scar tissue before and/or after treatment with a skin modification stimulus, for example within a few seconds, about 1 minute, or about 1 hour before and/or after treating the skin with a skin modification stimulus treatment, to within about 6 hours, about 12 hours, about 18 hours, or about 24 hours before and/or after treating the skin with a scar modification procedure. In various embodiments, the at least one C-glycoside or derivative thereof may be applied to the skin or scar tissue one or more times per day, for example 2 times per day, 3 times per day, or more. In further embodiments, the at least one C-glycoside or derivative thereof can be applied to the skin or scar tissue at least once a day over a period of one or more consecutive days, for example at least 2 days, at least 3 days, at least 4 days, at least 5 days, or more, or at least once a day over a period of one or more non-consecutive days, for example every other day, about three times a week, about twice a week, about once a week, about once every 2 weeks, about once every 3 weeks, about once every 4 weeks, or more. In further embodiments, methods according to the disclosure may be repeated one or more times, for example every day, every other day, about three times a week, about twice a week, about once a week, about once every 2 weeks, about once every 3 weeks, about once every 4 weeks, or more.

In still further embodiments, the methods comprise:
(1) treating skin or scar tissue with at least one type of skin modification stimulus, for example a laser, radiofrequency, an electrical, heat, a low-level light, a needle, or an abrasive instrument; and
(2) applying a composition comprising from about 0.01% to about 50%, from about 0.025% to about 40%, from about 0.05% to about 30%, from about 0.1% to about 20%, from about 0.2% to about 15%, from about 0.3% to about 12%, from about 0.4% to about 11%, from about 0.5% to about 10%, from about 0.6% to about 9%, from about 0.7% to about 8%, from about 0.8% to about 7%, from about 0.9% to about 6%, from about 1% to about 5%, or from about 2% to about 4% of at least one C-glycoside or derivative thereof to the treated skin or scar tissue, at least one time, preferably at least 2, 3, 4, 5, 6, 7, 8, 9, 10, 11, 12, 13, 14, 15, 16, 17, 18 ,19, 20, 21, 22, 23, 24, 25, 26, 27, 28, 29, or 30 times after treating the skin or scar tissue, wherein the at least one C-glycoside or derivative thereof is applied at least 12 hours apart, wherein all amounts are by weight, relative to the total weight of the composition in which it is present.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Implementation of the present technology will now be described, by way of example only, with reference to the attached figures.

FIG. 1 discloses a diagram of an exemplary scar revision surgery according to the disclosure which involves the use of at least one C-glycoside or derivative thereof. A scar is formed after tissue injury. The scar tissue is treated with an aesthetic laser, followed by hydroxypropyl tetrahydropyrantriol.

FIG. 2 discloses hematoxylin and eosin (H&E) stained cross-sections of Softskin™ model epidermal tissues that have (“Laser”) or have not (“Control”) been subjected to laser ablation. Some tissues were treated once a day for 3 days with 3% hydroxypropyl tetrahydropyrantriol in 3% propylene glycol (“Laser/3% Proxylane”), or 2% hyaluronic acid (“Laser/2% Hyaluronic Acid”). Cross-sections were obtained and stained on days 0, 2, and 4.

FIG. 3 discloses filament aggregating protein (Filaggrin) antibody immunostained cross-sections of Day 2 Softskin™ model epidermal tissues that a) have not been treated (control), b) were laser-treated, c) were laser treated, then treated with 3% hydroxypropyl tetrahydropyrantriol in 3% propylene glycol, and d) were laser treated, then treated with 3% propylene glycol control vehicle. Green: Filaggrin, Blue: DAPI (nucleus).

FIG. 4 discloses relative Filaggrin expression in day 2 Softskin™ model epidermal tissues before and after laser ablation, and in tissues further treated with 3% propylene glycol (negative control/placebo) vehicle or 3% hydroxypropyl tetrahydropyrantriol in 3% propylene glycol as measured using Image J software.

FIG. 5 discloses Ki-67 antibody immunostained cross-sections of Day 2 Softskin™ model epidermal tissues that a) have not been treated (control), b) were laser-treated, c) were laser treated, then treated with 3% hydroxypropyl tetrahydropyrantriol in 3% propylene glycol, and d) were laser treated, then treated with 3% propylene glycol control vehicle. Red: KI67, Blue: DAPI (nucleus).

FIG. 6 discloses the ratio of Ki67 (Antigen KI-67, or MKI67) expression to DAPI expression in day 2 Softskin™ model epidermal tissues before and after laser ablation, and in tissues further treated with 3% propylene glycol (negative control/placebo) vehicle or 3% hydroxypropyl tetrahydropyrantriol in 3% propylene glycol as measured using a 3D HISTECH PANNORAMIC MIDI II fluorescence scanner.

FIG. 7 discloses relative alpha Dickkopf WNT signaling pathway inhibitor 1 (DKK-1) expression in day 1 Softskin™ model epidermal tissues before and after laser ablation, and in tissues further treated with 3% propylene glycol (negative control/placebo) vehicle or 3% hydroxypropyl tetrahydropyrantriol in 3% propylene glycol.

FIG. 8 discloses relative alpha-smooth muscle actin (α-SMA) expression in day 2 Softskin™ model epidermal tissues before and after laser ablation, and in tissues further treated with 3% propylene glycol (negative control/placebo) vehicle or 3% hydroxypropyl tetrahydropyrantriol in 3% propylene glycol.

### DETAILED DESCRIPTION

The disclosure relates to compositions and methods for treating scar tissue, such as by increasing, accelerating, and/or improving healthy skin renewal instead of scar tissue, and/or improving the appearance of scarred skin. The type of scars that can be treated is not limited, and includes, for example, keloid scars, flat scars, raised scars, contracture scars, atrophic scars, stretch marks, etc. The disclosure also relates to compositions and methods for preventing or minimizing the formation of scar tissue prior to a skin injury, for example prior to a surgical procedure.

The compositions according to various aspects of the disclosure comprise at least one C-glycoside or derivative, for example at least one C-xylopyranoside or derivative, and in at least certain embodiments comprise hydroxypropyl tetrahydropyrantriol.

The methods according to various embodiments comprise treating scar tissue with at least one skin modification stimulus and applying at least one C-glycoside or derivative thereof, for example a C-xylopyranoside or derivative thereof, e.g. hydroxypropyl tetrahydropyrantriol, to the scar tissue. In other embodiments, the methods comprise treating uninjured skin with at least one skin modification stimulus and applying at least one C-glycoside or derivative thereof, for example a C-xylopyranoside or derivative thereof, e.g. hydroxypropyl tetrahydropyrantriol, to the skin, in order to prevent or reduce the formation of scars.

Without intending to be limited by theory, it is understood that C-glycosides and derivatives thereof may advantageously prevent or reduce the formation of scars, and/or may improve the appearance of scars, by reducing the conversion of fibroblasts to myofibroblasts and/or by targeting TGF-β1 signaling, which is reduced by modulating beta catenin (β-catenin) via Dickkopf WNT signaling pathway inhibitor 1 (DKK-1) expression, and/or enhances scar reduction kinetics. Thus, in some embodiments, the at least one C-glycoside or derivative thereof increases the expression level of DKK-1 in scar tissue, which targets Wnt/LRP6 association, increases the phosphorylation of β-catenin, and ultimately downregulates the signal transduction of TGF-β in scar tissue.

As such, compositions and methods according to the disclosure address the problem of scar prevention and treatment in a new and previously unknown manner, via a mechanism that is different from that of known methods such as retinoids, topical or injectable steroids, silicone dressings, and surgical and non-surgical medical procedures. By treating skin and/or scar tissue with a skin modification stimulus and applying at least one C-glycoside or derivative thereof to the targeted area, it encourages new growth of normal skin tissue, rather than new growth of scar tissue.

Surprisingly, it has been found that in some embodiments, the scar treatment and prevention methods and compositions according to the disclosure impart broader and/or stronger scar treatment and prevention benefits to skin, with or without enhanced kinetics, as compared to known scar treatments.

A more detailed description of compositions, methods, and kits for treating skin and scar tissue is provided below.

I. Compositions

Compositions according to the disclosure comprise at least one C-glycoside or derivative thereof. The compositions may further comprise additional components such carriers, active ingredients other than C-glycosides or derivatives thereof, and additives typically found in skin care compositions.

A C-glycoside is a sugar moiety linked via a carbon-carbon (C—C) bond to a non-sugar moiety (aglycone). In various embodiments, the C-glycoside may be chosen from C13-D-xylopyranoside-n-propan-2-one; Cα-D-xylopyranoside n-propan-2-one; C13-D-xylopyranoside-2-hydroxypropane; Cα-D-xylopyranoside-2-hydroxypropane; 1-(C13-D-fucopyranoside)-propane-2-one; 1-(Cα-D-fucopyranoside)-propan-2-one; 1-(C13-L-fucopyranoside)-propan-2-one; 1-(Cα-L-fucopyranoside)-propane-2-one; 2-one,1-(C13-D-fucopyranoside)-2-hydroxypropane, 1-(Cα-D-fucopyranoside)-2-hydroxypropane; 1-(C13-L-fucopyranoside)-2-hydroxypropane; 1-(Cα-L-fucopyranoside)-2-hydroxypropane; 1-(C13-D-glucopyranosyl)-2-hydroxylpropane; 1-(Cα-D-glucopyranosyl)-2-hydroxylpropane; 1-(C13-D-galactopyranosyl)-2-hydroxylpropane; 1-(Cα-D-galactopyranosyl)-2-hydroxylpropane-1-(C13-D-fucofuranosyl)propan-2-one; 1-(Cα-D-fucofuranosyl)-propan-2-one ; 1-(C13-L-fucofuranosyl)-propan-2-one; 1-(Cα-L-fucofuranosyl)-propan-2-one; C13-D-maltopyranoside-n-propan-2-one-Cα-D-maltopyranoside-n-propan-2-one-C13-D-maltopyranoside-2-hydroxypropane; Cα-D-maltopyranoside-2-hydroxypropane; as well as derivatives thereof or mixtures thereof.

In various embodiments, the at least one C-glycoside is a C-xylopyranoside. In at least some embodiments, the C-glycoside comprises, consists essentially of, or consists of C13-D-xylopyranoside-2-hydroxypropane, Cα-D-xylopyranoside-2-hydroxypropane, or mixtures thereof. In yet further embodiments, the C-glycoside comprises, consists essentially of, or consists of C13-D-xylopyranoside2-hydroxypropane (also known as hydroxypropyl tetrahydropyrantriol or Proxylane). Hydroxypropyl tetrahydropyrantriol has the structure of Formula (I) below:

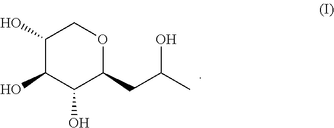

According to various embodiments, compositions according to the disclosure may comprise a total amount of C-glycoside and derivatives thereof from about 0.01% to about 30%, from about 0.05% to about 25%, from about 0.1% to about 20%, from about 0.2% to about 15%, from about 0.3% to about 12%, from about 0.4% to about 11%, from about 0.5% to about 10%, from about 0.6% to about 9%, from about 0.7% to about 8%, from about 0.8% to about 7%, from about 0.9% to about 6%, from about 1% to about 5%, from about 1.5% to about 4.5%, from about 2% to about 4%, from about 2.5% to about 3.5%, or about 3% by weight, including all ranges and subranges therebetween using any of the disclosed lower limits as a lower limit and disclosed upper limits as an upper limit, relative to the total weight of the composition in which it is present. For example, the total amount of C-glycoside and derivatives thereof in compositions according to the disclosure may be about 0.01%, about 0.05%, about 0.1%, about 0.25%, about 0.5%, about 0.75%, about 1° A), about 1.25%, about 1.5%, about 1.75%, about 2%, about 2.25%, about 2.5%, about 2.75%, about 3%, about 3.25%, about 3.5%, about 3.75%, about 5%, about 5.25%, about 5.5%, about 5.75%, about 6%, about 6.25%, about 6.5%, about 6.75%, about 7%, about 7.25%, about 7.5%, about 7.75%, about 8%, about 8.25%, about 8.5%, about 8.75%, about 9%, about 9.25%, about 9.5%, about 9.75%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, about 20%, about 21%, about 22%, about 23%, about 24%, about 25%, about 26%, about 27%, about 28%, about 29%, or about 30% by weight, including all ranges and subranges therebetween using any of the disclosed amounts as a lower limit or upper limit, relative to the total weight of the composition in which it is present.

The compositions will generally comprise one or more cosmetically-acceptable carriers, including but not limited to water and/or non-aqueous solvents. Exemplary non-aqueous solvents include glycerin; glycols, such as ethylene glycol, diethylene glycol, triethylene glycol, tetraethylene glycol, pentaethylene glycol, propylene glycol, dipropylene glycol, tripropylene glycol, polyethylene glycol, caprylyl glycol, and hexylene glycol; glycol ethers such as ethylene glycol monomethyl ether, ethylene glycol monoethyl ether, ethylene glycol monobutyl ether, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; and alcohols such as ethanol, propanol, isopropanol, butanol, pentanol, hexanol, heptanol, octanol, decanol, dodecanol, hexadecanol, octadecanol, eicosadecanol, 2-propanol, 2-methyl-1-propanol, 2-methyl-2-butanol, cetyl alcohol, stearyl alcohol, arachidyl alcohol, behenyl alcohol, and cyclohexanol. Mixtures of two or more non-aqueous solvents may also be chosen, and it is contemplated that in various embodiments, the carrier comprises water and at least one non-aqueous solvent.

The compositions may optionally comprise one or more active ingredients other than C-glycosides or derivatives thereof, such as, for example, actives that can modulate inflammation (including but not limited to carotenoids, curcumin, steroids, cannabinoids, glycoproteins, essential oils, flavonoids & flavonoid derivatives, phenolic acids, ascorbic acid, polyphenols, marine and algal extracts), actives that can enhance skin barrier (including but not limited to pantothenic acid, ceramides, pseudo ceramides such as 2-Oleyl-1,3-octadecanediol, niacinamides, niacinamide derivatives, carob seed extracts, oligo-galactomannan, colloidal oatmeal, hyaluronic acids, probiotics and 3-glucan), and/or actives that can modulate skin fibrosis (including but not limited to inhibitors of TNF, inhibitors of TGF-β, inhibitors of mTOR pathway and kynurenic acid and its derivatives).

The compositions may optionally contain one or more additives such as those typically found in the skin care compositions, including but not limited to oils, waxes, or other fatty substances; gelling agents and/or thickeners; emulsifiers; moisturizing agents; emollients; sunscreens; hydrophilic or lipophilic active agents, such as ceramides; agents for combatting free radicals; bactericides; sequestering agents; preservatives; pH adjusters; skin penetration enhancers; fragrances; surfactants; fillers; natural products or extracts thereof, such as aloe or green tea extract; vitamins; and/or coloring materials. The amount of such additives, if present, will vary depending on the intended use and/or properties of the composition and the effect desired, but will, in individual or combined amount, typically range from about 0.0001% to about 20%, such as from about 0.001% to about 15%, from about 0.01% to about 10%, from about 0.1% to about 7.5%, from about 0.5% to about 5%, or from about 1% to about 3% by weight, relative to the total weight of the composition.

The form of the compositions according to the disclosure is not limited, and may include, for example, liquids, emulsions, suspensions, lotions, creams, gels (including hydrogels), balms, pastes, serums, salves, foams, or the like.

The compositions are typically applied topically and can, in various embodiments, be applied by the consumer as an at-home formulation, or by a professional, e.g. in a clinical setting as part of an aesthetic procedure.

A non-limiting example of a composition according to the disclosure includes a carrier, for example water and/or at least one non-aqueous solvent, optionally at least one active ingredient other than C-glycosides or derivatives thereof, and/or at least one additive, and a total amount of C-glycosides and derivatives thereof ranging from about 0.01% to about 30%, from about 0.05% to about 25%, from about 0.1% to about 20%, from about 0.2% to about 15%, from about 0.3% to about 12%, from about 0.4% to about 11%, from about 0.5% to about 10%, from about 0.6% to about 9%, from about 0.7% to about 8%, from about 0.8% to about 7%, from about 0.9% to about 6%, from about 1% to about 5%, from about 1.5% to about 4.5%, from about 2% to about 4%, from about 2.5% to about 3.5%, or about 3% by weight, including all ranges and subranges therebetween, relative to the total weight of the composition in which it is present. A further non-limiting example of a composition according to the disclosure includes a carrier comprising water and optionally at least one non-aqueous solvent, optionally at least one active ingredient other than C-glycosides or derivatives thereof and/or at least one additive, and hydroxypropyl tetrahydropyrantriol present in an amount ranging from about 0.01% to about 30%, from about 0.05% to about 25%, from about 0.1% to about 20%, from about 0.2% to about 15%, from about 0.3% to about 12%, from about 0.4% to about 11%, from about 0.5% to about 10%, from about 0.6% to about 9%, from about 0.7% to about 8%, from about 0.8% to about 7%, from about 0.9% to about 6%, from about 1% to about 5%, from about 1.5% to about 4.5%, from about 2% to about 4%, from about 2.5% to about 3.5%, or about 3% by weight, including all ranges and subranges therebetween, relative to the total weight of the composition in which it is present.

II. Methods

In various embodiments, the disclosure relates to methods of treating scar tissue to reduce or improve the appearance of the scar. In further embodiments, the disclosure relates to methods of treating skin, such as skin that does not have visible scar tissue, to prevent and/or reduce the formation of scar tissue, for example uninjured skin. In yet further embodiments, the disclosure relates to methods of reducing the conversion of fibroblasts to myofibroblasts, targeting transforming growth factor beta 1 (TGF-β1) signaling, reducing modulating beta catenin (β-catenin), increasing the expression level of DKK-1, disrupting Wnt/LRP6 association, increasing the phosphorylation of p-catenin, and/or downregulating the signal transduction of TGF-β.

Typically, the methods comprise applying at least one C-glycoside or derivative thereof, for example a C-xylopyranoside or derivative thereof, e.g. hydroxypropyl tetrahydropyrantriol, to a desired area to be treated, for example to uninjured skin or to scar tissue (referred to collectively in some instances herein as a “target area”), before, during, and/or after treating the uninjured skin or scar tissue with a stimulus for modifying skin or scar tissue (referred to interchangeably herein as a “skin modification stimulus”). In various methods, the C-glycoside or derivative thereof comprises, consists essentially of, or consists of C-xylopyranosides and/or derivatives thereof, and in further methods, the C-glycoside or derivative thereof comprises, consists essentially of, or consists of hydroxypropyl tetrahydropyrantriol.

In methods according to the disclosure, useful skin modification stimuli include those used in the skin care field to modify or damage skin or scar tissue in order to encourage new growth, generally in a controlled manner. These treatments typically damage the outermost layers of the skin or scar tissue to a relatively mild degree, which sets into motion the body's natural healing process. Such treatments may also stimulate the production of collagen. Thus, any known method or device for modifying or damaging the skin or scar tissue can be used in the methods according to the disclosure. For example, in various embodiments, the skin modification stimulus may modify or damage the skin or scar tissue by cutting, burning, chemically damaging, ablating, micro-ablating, coagulating, or otherwise affecting the corneal layer and subcutaneous tissue of the target area. Therefore, as used herein, “modified scar tissue” is scar tissue that has been modified or damaged with one or more skin modification stimuli, and is healing from treatment with the skin modification stimulus. Likewise, “modified skin tissue” is skin tissue, typically uninjured and/or without scar tissue, that has been modified or damaged with one or more skin modification stimuli, and is healing from treatment with the skin modification stimulus.

The skin modification stimulus used in the methods according to the disclosure may, in various embodiments, be invasive or non-invasive. By way of non-limiting example, the skin modification stimulus can be a laser, plasma, radiofrequency, a high-intensity focused ultrasound, an electrical field, heat, a low-level light device, a needle, a microneedle, an abrasive element, a chemical agent such as a chemical exfoliating agent, or the like, and the skin modification stimulus treatment may be a laser procedure, a microneedling procedure, a high-intensity focused ultrasound procedure, an electroporation procedure, a dermabrasion procedure, a microdermabrasion procedure, a plasma skin rejuvenation procedure, a chemical exfoliation procedure, or the like.

Any variety of cosmetic lasers, distinguished by their wavelength and their mode of delivering the laser, can be used. In various embodiments, the target area may be treated with ablative or non-ablative lasers. Ablative lasers may include, e.g., carbon dioxide or erbium lasers. Non-ablative lasers may include, e.g., pulsed light, pulsed-dye, and fractional lasers. Lasers can also be modified when combined with other elements, such as gases, precious stones, and metals. Nonlimiting examples include Clear+Brilliant® lasers, fractional photothermolysis lasers, alexandrite lasers, carbon dioxide (CO₂) lasers, erbium (Er:YAG) lasers, intense pulsed light (IPL) lasers, Nd:YAG lasers, pulse dye lasers, Q-switched lasers, picosecond lasers, etc.

In various embodiments, the target area may be treated with mechanical microneedling, which uses a motorized pen with tiny slender needles to create thousands of channels that are up to 2.5 millimeters deep. These channels disrupt the uninjured skin or scar tissue to allow for growth of new skin. Non-limiting examples of mechanical microneedling devices that can be used include SkinPen Precision, MDpen, Skin Stylus, etc.

In some embodiments, the target area may be treated with fractional radiofrequency (RF) microneedling, which is a heat-based therapy that uses thicker needles to transmit heat deep into the skin. RF microneedling thermally coagulates tissue to stimulate collagen denaturation and renewal. Radiofrequency (RF) microneedling generates less microchannels than mechanical needling. In an embodiment, the target area may be treated with standard or short-pulse RF microneedling. Non-limiting examples of RF devices that may be used include Scarlet SRF, Agnes RF, Endymed Intensif, Secret RF, Vivace, Virtue RF, Morpheus8, etc.

The target area can also be treated with ultrasound, such as through ultratherapy. Ultrasound can thermally loosen, stretch, and re-orient collagen tissue. Ultrasound mechanically acts on skin and scar tissue as a micro-massage and produces microscopic droplets of oxygen (calvitation) from the vibration.

Electroporation mesotherapy can also be utilized with the disclosed methods. Electroporation uses electricity to boost the permeability of the skin or scar tissue membrane by creating microscopic channels that open up pathways deep into the skin, thus disrupting scar tissue.

Dermabrasion, including microdermabrasion, may also be used to treat the target area in some embodiments. Dermabrasion involves the controlled deeper abrasion of the upper to mid layers of the uninjured skin or scar tissue with any variety of strong abrasive devices such as a wire brush, diamond wheel (or fraise), or serrated wheel. Exemplary embodiments may include crystal microdermabrasion or diamond microdermabrasion.

In other embodiments, plasma skin rejuvenation may be used in the disclosed methods. Plasma skin regeneration can be used to deliver energy in the form of plasma to the target area, resembling a thermal device and a dry peel. This process is useful in particular for breaking up scar tissue.

In certain embodiments, a chemical agent such as a chemical exfoliating or peeling agent (also known as chemexfoliation or derma-peeling) can be used as a skin modification stimulus. Exemplary and non-limiting chemical agents that may be used to treat the target area include chemical agents capable of acting either directly on peeling by encouraging exfoliation, such as β-hydroxyacids (BHAs), for example salicylic acid and derivatives thereof (including n-octanoyl 5-salicylic acid, otherwise known as capryloyl salicylic acid (INCI name)); α-hydroxyacids (AHAs), such as glycolic, lactic, tartric, malic, or mandelic acids; 8-hexadecene-1,16-dicarboxylic acid, or 9-octadecene dioic acid; urea and derivatives thereof; trichloroacetic acid; gentisic acid and derivatives thereof; oligofuctoses; cinnamic acid; extract of Saphora japonica; resveratrol; resorcinol; carbolic acid (phenol); or mixtures thereof.

Further exemplary and non-limiting chemical peeling agents that may be used include compounds involved in peeling or degrading corneodesmosomes, such as aminosulfonic compounds, e.g. 4-(2-hydroxyethyl)piperazine-1-propanesulfonic acid (HEPES); 2-oxothiazolidine-4-carboxylic acid (procysteine) and derivatives thereof; the derivatives of a-amino acids of the glycine type (as described in EP-0 852 949) and also sodium methyl glycine diacetate sold by BASF under the trade name Trilon M; honey; derivatives of sugar such as O-octanoyl-6-D-maltose and N-acetyl glucosamine; or mixtures thereof.

As still further exemplary and non-limiting chemical peeling agents suitable for use in compositions according to the disclosure, mention be made of EDTA and derivatives thereof, laminaria extracts, O-linoleyl-6-D-gluocose; (3-hydroxy-2-pentylcyclopentyl) acetic acid, glycerol trilactate, S carboxymethyl cysteine, silica-containing derivatives of salicylate such as those described in patent EP 0 796 861, oligofuctases such as those described in patent EP 0 218 200, agents having effects on transglutaminase as in patent EP 0 899 330; extract of the flower ficus opuntia indica such as Exfolactive® from Silab; 8-hexadecene 1,16-dicarboxylic acid; esters of glucose and of vitamin F; or mixtures thereof.

The chemical agent(s) may, in certain embodiments, be chosen from a-hydroxy acids such as citric, lactic, glycolic, malic, tartric, or mandelic acids; β-hydroxy acids such as salicylic acid or derivatives thereof, e.g. n-octanoyl-5-salicylic acid; tricholoacetic acid; phenols; croton oil peels; or mixtures thereof.

The C-glycoside or derivative thereof will typically remain on the skin (e.g. uninjured skin or scar tissue, which may or may not be modified skin tissue or modified scar tissue) for a time sufficient to achieve the desired benefits. Thus, it may be preferable to leave the C-glycoside or derivative thereof on the skin until it penetrates therethrough or is removed in the ordinary course of personal hygiene, e.g. the next time the skin is washed. For example, in some embodiments, the at least one C-glycoside or derivative thereof is left on the skin for a desired period of time, for example at least 10 seconds, at least 20 seconds, at least 30 seconds, at least 40 seconds, at least 50 seconds, at least 1 minute, at least 2 minutes, at least 3 minutes, at least 5 minutes, at least 10 minutes, at least 15 minutes, at least 20 minutes, at least 25 minutes, at least 30 minutes, at least 35 minutes, at least 40 minutes, at least 45 minutes, at least 1 hour, at least 2 hours, at least 5 hours, at least 8 hours, at least 10 hours, at least 15 hours, at least 20 hours, at least 24 hours, at least 36 hours, or at least 48 hours before it is removed, such as by, for example, wiping or rinsing the skin. For example, the at least one C-glycoside or derivative thereof may be left on the skin for a period of time ranging from 5 minutes to 60 minutes, such as from 5 minutes to 50 minutes, from 5 minutes to 40 minutes, from 5 minutes to 35 minutes, from 5 minutes to 30 minutes, from 5 minutes to 25 minutes, from 5 minutes to 20 minutes, from 5 minutes to 15 minutes, or from 5 minutes to 10 minutes. As a further example, the at least one C-glycoside or derivative thereof may be left on the skin for a period of time ranging from 10 minutes to 60 minutes, such as from 10 minutes to 50 minutes, from 10 minutes to 40 minutes, from 10 minutes to 35 minutes, from 10 minutes to 30 minutes, from 10 minutes to 25 minutes, from 10 minutes to 20 minutes, or from 10 minutes to 15 minutes.

The step of applying a C-glycoside or derivative thereof may comprise one or more applications, such as two or more applications, three or more applications, etc., to the target area. Thus, in various embodiments, the methods comprise applying at least one C-glycoside or derivative thereof to the target area at least one time before, at least one time during, and/or at least one time after treatment with the skin modification stimulus. For example, the at least one C-glycoside or derivative thereof may be applied to the target area at least one time before, at least one time during, and at least one time after treatment with the skin modification stimulus treatment. As another example, the methods comprise applying at least one C-glycoside or derivative thereof to the target area at least two times before, at least two times during, and/or at least two times after treatment with the skin modification stimulus. As yet a further example, the methods comprise applying at least one C-glycoside or derivative thereof to the target area at least one time, such as at least two times, before treatment with the skin modification stimulus, and at least one time, such as at least two times, after treatment with the skin modification stimulus. As a still further example, the methods comprise applying at least one C-glycoside or derivative thereof to the target area at least one time, such as at least two times, after treatment with the skin modification stimulus.

In various embodiments, the at least one C-glycoside or derivative thereof can be applied to the target area at least one time per day, at least two times per day, or at least three times per day, such as from one to five times a day, from one to four times a day, from one to three times a day, or from one to two times a day, for example once per day, before and/or after treatment with the skin modification stimulus.

The at least one C-glycoside or derivative thereof can be applied to the target area at least one time per day, for at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, or about 60 consecutive or non-consecutive days, before and/or after treatment with the skin modification stimulus. The at least one C-glycoside or derivative thereof may be applied to the target area at least one time per day, for at least about 1, about 2, about 3, about 4, about 5, about 6, about 7 days per week. For example, the at least one C-glycoside or derivative thereof may be applied to the target area at least two times per day, wherein the second or subsequent application is at least about 6 hours, for example at least about 8 hours, at least about 10 hours, at least about 12 hours, or at least about 15 hours after the prior application.

In various embodiments, the methods according to the disclosure may be performed at least one time per month for at least 1, 2, 3, 4, 5, 6, 7, 8, 9, 10, 11, 12, 13, 14, 15, 16, 17, 18, 19, 20, 21, 22, 23, 24, 25, 26, 27, or 28 consecutive or non-consecutive months.

In various embodiments, the at least one C-glycoside or derivative thereof is applied to the target area within about 1 minute, such as within about 5 minutes, within about 10 minutes, within about 15 minutes, within about 20 minutes, within about 30 minutes, within about 45 minutes, within about 60 minutes, within about 90 minutes, within about 2 hours, within about 3 hours, within about 4 hours, within about 5 hours, within about 6 hours, within about 8 hours, within about 10 hours, within about 12 hours, within about 18 hours, within about 24 hours, within about 36 hours, or within about 48 hours before and/or after treating the skin or scar tissue with a skin modification stimulus treatment.

In various methods according to the disclosure, applying at least one C- glycoside or derivative thereof is accomplished by applying a composition comprising at least one C-glycoside or derivative thereof, for example a composition comprising at least one C-xylopyranoside or derivative thereof, e.g. hydroxypropyl tetrahydropyrantriol, to the target area before, during, and/or after the skin or scar tissue is treated with a skin modification stimulus. Any of the compositions described herein may be used in the methods of the disclosure.

In at least some embodiments, treatments with the skin modification stimulus are carried out with at-home devices. As non-limiting examples, microneedling and microdermabrasion can be accomplished with at-home devices. Such at-home devices can be sold in kits with compositions comprising at least one C-glycoside or derivative thereof, for example a composition comprising at least one C-xylopyranoside or derivative thereof, e.g. hydroxypropyl tetrahydropyrantriol.

The methods according to the disclosure treat skin or scar tissue using the skin modification stimuli according to known, typical procedures. Thus, although it is possible to do so, it is not necessary to vary the length, intensity, or other parameters of treatments with the skin modification stimuli from those which are currently performed in order to obtain the surprising and unexpected scar prevention benefits and/or improvement or reduction in scar appearance according to the disclosure. For example, in a typical microdermabrasion procedure, a device having exfoliating crystals is moved across the skin with light pressure for a period of about 30 minutes. In a method according to the disclosure where an abrasive element is chosen as a skin modification stimulus, the same microdermabrasion procedure can be performed and the at least one C-glycoside or derivative thereof can be applied to the target area before, during, and/or after the procedure.

The disclosed methods do not relate to enhancing or improving wound healing, but rather to the separate and distinct biological processes related to scar formation discussed herein. Thus, in at least some embodiments, the methods according to the disclosure do not include applying a C-glycoside or derivative thereof to wounded skin that does not have scar tissue. In other embodiments, the methods according to the disclosure do not include applying a C-glycoside or derivative thereof to broken skin. In still further embodiments, the methods according to the disclosure do not include applying a C-glycoside or derivative thereof to wounded skin that has healed for fewer than seven (7) days.

According to various embodiments of the disclosure, the methods comprise treating scar tissue by applying at least one C-glycoside or derivative thereof to the scar tissue in connection with treating the scar tissue with a skin modification stimulus. In various embodiments, the methods comprise:
(1) treating scar tissue with at least one skin modification stimulus,
(2) applying at least one C-glycoside or derivative thereof to the scar tissue.

In some embodiments, the step of (1) treating scar tissue with at least one skin modification stimulus occurs before the step of (2) applying at least one C-glycoside or derivative thereof to the scar tissue, such that the at least one C-glycoside or derivative thereof is applied to modified scar tissue. In some embodiments, the step of (2) applying at least one C-glycoside or derivative thereof to the scar tissue comprises applying a composition comprising at least one C-glycoside or derivative thereof and at least one carrier to the scar tissue before and/or after step (1), wherein the total amount of C-glycosides and derivatives thereof present in the composition ranges from about 0.1% to about 30%, for example about 0.5% to about 10%, about 0.75% to about 8%, about 1 to about 6%, about 1.25% to about 5%, about 1.5% to about 4%, about 2% to about 3.5%, or about 2.5% to about 3.5% by weight, relative to the total weight of the composition.

According to further embodiments of the disclosure, the methods comprise applying at least one C-glycoside or derivative thereof to skin prior to a skin injury, e.g. prior to a surgical procedure in connection with treating the skin with a skin modification stimulus. In various embodiments, the methods comprise:
(1) treating uninjured skin with at least one skin modification stimulus, and
(2) applying at least one C-glycoside or derivative thereof to the skin.

In some embodiments, the step of (1) treating uninjured skin with at least one skin modification stimulus occurs before the step of (2) applying at least one C-glycoside or derivative thereof to the uninjured skin, such that the at least one C-glycoside or derivative thereof is applied to modified skin tissue. In some embodiments, the step of (2) applying at least one C-glycoside or derivative thereof to the uninjured skin comprises applying a composition comprising at least one C-glycoside or derivative thereof and at least one carrier to the uninjured skin before and/or after step (1), wherein the total amount of C-glycosides and derivatives thereof present in the composition ranges from about 0.1% to about 30%, for example about 0.5% to about 10%, about 0.75% to about 8%, about 1% to about 6%, about 1.25% to about 5%, about 1.5% to about 4%, about 2% to about 3.5%, or about 2.5% to about 3.5% by weight, relative to the total weight of the composition.

Optionally, in some embodiments, one or more additional active agents other than C-glycosides or derivatives thereof can be applied to the skin either as part of a composition comprising the at least one C-glycoside or derivative thereof or separately, including by way of example actives that can modulate inflammation (including but not limited to carotenoids, curcumin, steroids, cannabinoids, glycoproteins, essential oils, flavonoids & flavonoid derivatives, phenolic acids, ascorbic acid, polyphenols, marine and algal extracts) actives that can enhance skin barrier (including but not limited to pantothenic acid, ceramides, pseudo ceramides such as 2-Oleyl-1,3-octadecanediol, niacinamides, niacinamide derivatives, carob seed extracts, oligo-galactomannan, colloidal oatmeal, hyaluronic acids, probiotics and β-glucan), and/or actives that can modulate skin fibrosis (including but not limited to inhibitors of TNF, inhibitors of TGF-β, inhibitors of mTOR pathway and kynurenic acid and its derivatives). The additional active agent(s) may be applied to the target area before, during, and/or after the uninjured skin or scar tissue is treated with a skin modification stimulus, and/or before, during, and/or after the at least one C-glycoside or derivative thereof is applied to the target area.

In some embodiments, at least one additional composition, such as, for example, moisturizer, sunscreen, and/or anti-aging compositions, may be applied to the target area before and/or after application of the at least one C-glycoside or derivative thereof. In various embodiments, the at least one C-glycoside or derivative thereof is applied to the target area first, and is not removed prior to application of at the least one additional skin composition. In another embodiment, the at least one additional skin composition applied to the target area first, and is not removed prior to application of the at least one C-glycoside or derivative thereof.

If desired, any of the disclosed steps or methods may be repeated one or more times, using the same or a different skin modification stimulus, and/or the same or a different C-glycoside or derivative. If different, treatment with a skin modification stimulus may be different in any manner, for example may use different skin modification stimuli, may use the same stimulus but for a different length of time, etc. Likewise, if different, application of a C-glycoside or derivative may be different in any manner, for example may be different C-glycosides, may be the same C-glycoside but at different concentrations, etc.

For example, one method may comprise repeating treatment with the skin modification stimulus one or more times before and/or after the at least one C-glycoside or derivative thereof is applied to the target area, where the treatments with the skin modification stimulus may be the same or different. Another exemplary method may comprise repeating application of at least one C-glycoside or derivative thereof to the target area one or more times before and/or after treatment with the skin modification stimulus, where the C-glycoside or derivative may be the same or different.

As another non-limiting example, a first skin modification stimulus may be a laser, where the first treatment is a Clear+Brilliant® laser procedure, and a second skin modification stimulus may be an abrasive element, where the second treatment is a microdermabrasion procedure. As yet another non-limiting example, a first skin modification stimulus may be a laser, where the first treatment is a Clear+Brilliant® laser procedure, and a second treatment may be a second Clear+Brilliant® laser procedure.

In another example still, a first step of applying at least one C-glycoside or derivative thereof to skin of an individual prior to surgery before, during, and/or after treating the skin with at least one skin modification stimulus; and then, after the individual's surgical wound heals for 7 days, a second step of applying at least one C-glycoside or derivative thereof to scar tissue formed at the site of the wound before, during, and/or after treating the scar tissue with at least one skin modification stimulus, wherein either or both of the C-glycoside or derivative thereof and the skin modification stimulus in the first and second steps are the same or different.

As a further non-limiting example, a method may be a method of treating scar tissue in order to reduce or improve the appearance thereof, where a first skin modification stimulus is a laser and the first treatment is a Clear+Brilliant® laser procedure, and a second skin modification stimulus is an abrasive element where the second treatment is a microdermabrasion procedure, with application of at least one C- glycoside or derivative thereof to the target area before, between, during, and/or after the first and second treatments, where the C-glycoside or derivative in each application is the same or different.

A person skilled in the art will appreciate based on the foregoing that many additional variations in treatment regimens according to the disclosure are contemplated to be within the scope of the disclosure.

In this application, the use of the singular includes the plural unless specifically stated otherwise. Thus, the terms “a,” “an,” and “the” are understood to encompass the plural as well as the singular; thus, “a skin modification stimulus” should be understood to mean “at least one skin modification stimulus” unless expressly stated otherwise. In this application, the use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one unit unless specifically stated otherwise.

The term “and/or” should be understood to include both the conjunctive and the disjunctive and expressly covers instances of either without reference to the other. For example, applying a C-glycoside or derivative to a target area “before and/or after” treatment with a skin modification stimulus includes applying a C-glycoside or derivative to a target area before treatment with a skin modification stimulus and after treatment with a skin modification stimulus, as well as applying a C-glycoside or derivative to a target area before treatment with a skin modification stimulus or after treatment with a skin modification stimulus.

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%), such as within 9%, 8%, 7%, 6%, 5%, 4%, 3%, 2%, or 1%, according to various embodiments. All numbers (including ratios, concentrations, etc.) disclosed herein are understood to include the term “about” whether or not present.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, +/−1%, +/−0.5%, +/−0.1%, or +/−0.01%.

As used herein, the expressions “ranging from” and “between” are inclusive of the endpoints of the recited range(s).

As used herein, all ranges provided are meant to include every specific range within, and combination of sub ranges between, the given ranges. Thus, a range from 1-5, includes specifically 1, 2, 3, 4, and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The term “comprising” (and its grammatical variations) as used herein is used in the inclusive sense of “having” or “including” and not in the exclusive sense of “consisting only of.”

As used herein, “uninjured skin” means skin that has not been injured in a manner that could lead to a visible scar, such as, for example, a burn, a laceration, an abrasion, acne, a skin infection, an animal bite, a piercing, surgery, etc., but does not include skin that is damaged by treatment with a skin modification stimulus as described herein. Uninjured skin is intended to be distinct from skin that was previously injured, such as, for example, by a burn, a laceration, an abrasion, acne, a skin infection, an animal bite, a piercing, surgery, etc., and which, when treated according to methods of the disclosure, has visible scar tissue from such injury.

As used herein, “topical application” (and its grammatical variations) refers to the application of the compositions of the disclosure onto keratinous substrates such as skin.

As used herein, “reducing” or “improving” the appearance of a scar or scar tissue means that the scar or scar tissue is less visible or noticeable to the naked eye than it would have been in the absence of treatment according to methods disclosed herein.

As used herein, “preventing” the formation of a scar or scar tissue means preventing the appearance of scar tissue that is visible to the naked eye.

As used herein, “reducing” the formation of a scar or scar tissue means that although a scar or scar tissue may form, its appearance to the naked eye is less visible or noticeable than it would have been in the absence of treatment according to methods disclosed herein.

Unless otherwise expressly stated, it is in no way intended that any method set forth herein be construed as requiring that its steps be performed in a specific order. Accordingly, where a method claim does not expressly recite an order to be followed by its steps or it is not otherwise specifically stated in the claims or descriptions that the steps are to be limited to a specific order, it is no way intended that any particular order be inferred.

The compositions and methods of the present disclosure can comprise, consist of, or consist essentially of the essential elements and limitations of the disclosure described herein, as well as any additional or optional ingredients, components, or limitations described herein or otherwise useful.

Several embodiments having been described above, the disclosure may be better understood by reference to examples. The following examples are intended for illustration purposes only, and should not be construed as limiting in any way.

### EXAMPLES

The following examples are intended to be non-limiting and explanatory nature only.

### Example 1: Cell Cultures

Surgical skin samples were be taken from adults and subjected to the 0.25% trypsin dermis/epidermis separation method described in Rheinwald to obtain suspensions of normal human keratinocytes (NHK) and dermal fibroblasts. See Rheinwald J. G., Green H.: Serial cultivation of strains of human epidermal keratinocytes: The formation of keratinizing colonies from single cells. Cell. 6(3):331-43. 1975. The separated normal human dermal fibroblasts were cultured in DMEM+10% fetal calf serum. The fibroblasts were cast onto a lower layer of proto-SoftSkin™ gel. A full-thickness dermal layer equivalent was obtained after contraction of a mixture of Collagen Type I and human normal fibroblasts. The separated normal human keratinocyte cell cultures were initiated using the 3T3 feeder-layer technique described in Rheinwald J. G., Green H.: Epidermal growth factor and the multiplication of cultured human epidermal keratinocytes. Nature 265:421-424 (1977). The separated normal human keratinocytes were seeded on a lattice on the top layer of a proto-SoftSkin™ gel and the culture was left for 7 days in submerged conditions at 37° C. in the presence of 5% carbon dioxide in MEM containing 10% fetal calf serum, allowing the cells to proliferate and form an epidermal layer. Then, the culture was raised upwards to air-liquid interface for an additional 7 days to grow reconstructed skin models (SoftSkin™).

### Example 2: CO₂ Laser Skin Modification Stimulus

The reconstructed skin samples derived from normal human dermal fibroblasts and normal human keratinocytes were subjected to CO₂ laser treatment. Laser treatment was conducted using an ablative fractional CO₂RE® system (Syneron (Candela, Israel)). The energy level was set to Deep mode (fractional coverage 5%, core energy 30 mJ, core energy fluence 170 J/cm²). The tissues were treated with the laser for 0.5 second per wound, causing 9 mm wound patterns with 56 minor wounds with individual diameters of 200 um and depths of 700 um per well. After laser treatment (Day 0), skin models were transferred to 6-well plates. The media was changed on Day 0, Day 2 and Day 4.

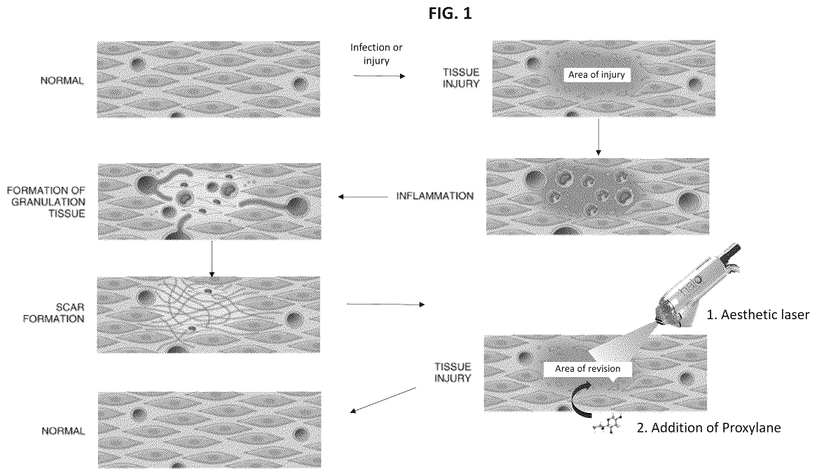

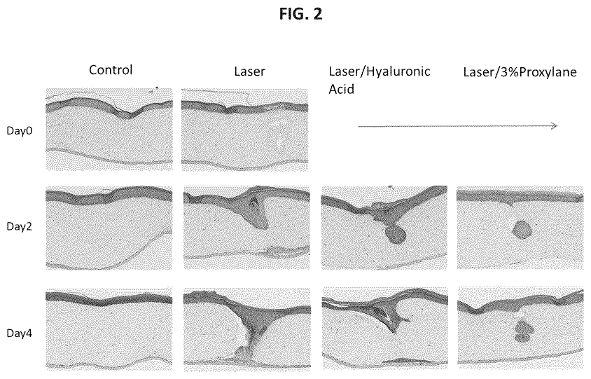

FIG. 1 illustrates the experimental setup, where the reconstructed skin is subjected to laser wounds which leave behind a series of well-defined circular wounds. The healing process is tracked by studying the healing of the circular wound over time. FIG. 2 discloses the wound recovery kinetics of Softskin™ model epidermal cells after laser ablation through hematoxylin and eosin (H&E) stained cross-sections. The “Control” and “Laser” cross-sections of FIG. 2 demonstrate that the laser wounded skin model is an acceptable model for simulating the process of wound healing, which includes inflammation, proliferation and remodeling.

Skin samples were harvested on Day 0, Day 2, Day 4 for tissue staining experiments and Day 2 for ELISA assay.

### Example 3: Proxylane Topical Application

35% Proxylane (C-glycoside) in 35% propylene glycol(vehicle) was diluted with water to prepare a working solution as 3% Proxylane in 3% propylene glycol. 100% of the propylene vehicle was diluted with water as a working solution of 3% propylene glycol vehicle as placebo treatment (negative control). A working solution of 3% hyaluronic acid (HA) in water was also prepared as a control to determine whether alternative actives can induce the same repair/scarring prevention effect as proxylane.

10 uL of the prepared working solutions were applied to the laser wound surface of separate skin models every 24 hours starting 2 hours post laser treatment on Day 0, 24 hours (Day 1), and 48 hours (Day 2). The impact of C-glycosides on the skin was observed by studying the impact of Proxylane on laser-induced wounds using reconstructed skin. The following Examples demonstrate that C-glycosides can be used to improve scar appearance and reduce scar formation.

### Example 4: Histology and Immunofluorescence Staining

10 mm diameter segments of reconstructed skin tissue was punched for histological staining. Skin samples were embedded in Optimal Cutting Temperature (OCT) compound for cryosection. 7 μm sectioned slices were cut and dried for 1 hour at room temperature, then fixed in cold acetone for 5 minutes, washed with DPBS twice, blocked with 0.2% BSA in DPBS for another 10 min.

Some of the fixed slices were stained with haematoxylin and eosin (H&E). Exemplary results are provided in FIG. 2. Keratinocytes invaded laser dermal injury sites in hydroxypropyl tetrahydropyrantriol treated tissues, but not in hyaluronic acid or negative control treated tissues. These immunostaining analysis from the wounded skin model further elucidate the benefits of C-glycosides for wound healing and scar management applications.

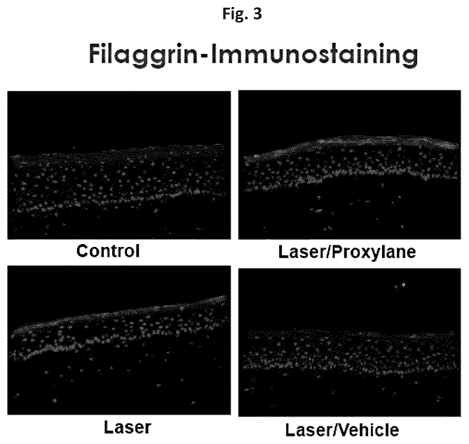

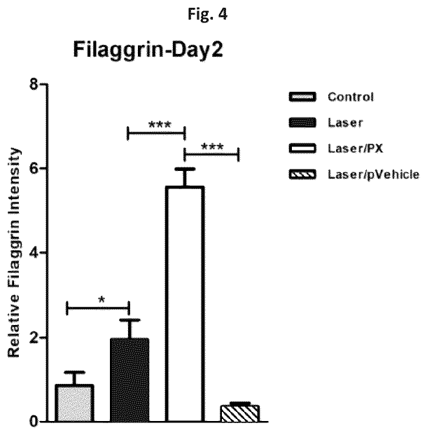

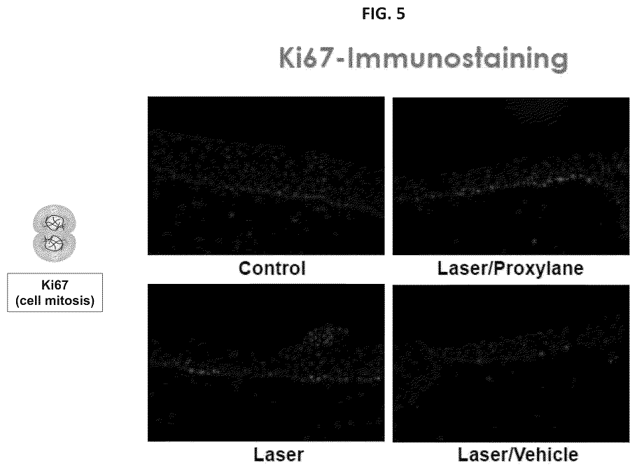

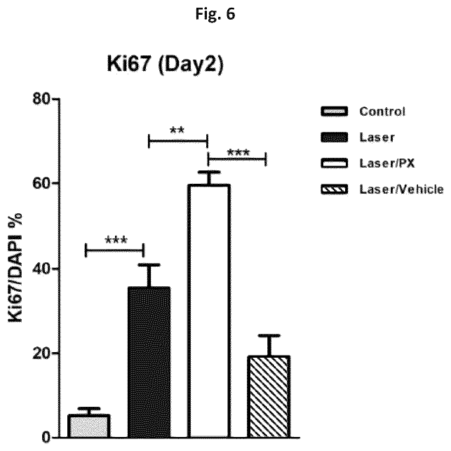

Some of the fixed slices were stained to assess the presence of Filaggrin or Ki67. Filaggrin (MA5-13440, Thermo Fisher Scientific, Waltham, MA, USA, mouse, 1:100) and Ki67 (M7240, Dako, Glostrup, Denmark, mouse 1:100) primary antibodies were separately diluted in DPBS containing 1% donkey serum and the sectioned slices were incubated with the Filaggrin or Ki67 antibodies at room temperature for 2 hours in humid chamber, then washed with DPBS three times. Secondary antibodies (donkey anti-mouse Alexa Fluor 488, Invitrogen A21202, 1:300) were diluted in DPBS containing 1% donkey serum, and the Filaggrin or Ki67 antibody-stained slices were incubated with the secondary antibodies for 1 hour at room temperature and washed with DPBS three times. The secondary antibody-stained slices were then stained with DAPI (Invitrogen D1306, 1:10000), then washed with DPBS. The stained slices were imaged with a 3D HISTECH PANNORAM IC MIDI II fluorescence scanner. Results are provided in FIGS. 3 and 5. The ratio of Ki67 positive cells/total cell number in basal layer was analyzed for each scanned image in the Ki67 antibody-stained slices. Relative fluorescence intensity of Filaggrin was measured in the Filaggrin antibody-stained slices using Image J software. Results are provided in FIGS. 4 and 6 respectively.

C-glycoside treated wounded skin showed significantly higher level of KI67 and filaggrin compared to wounded skin alone and placebo treated wounded skin (FIGS. 4 and 6). Hydroxypropyl tetrahydropyrantriol (3%) increased Filaggrin expression, which assists in skin barrier function and water retention, in a statistically significant manner compared to laser treatment alone, laser+vehicle treatment and the untreated control. Hydroxypropyl tetrahydropyrantriol (3%) increased Ki67 expression, a marker for keratinocyte proliferation, in a statistically significant manner compared to laser treatment alone, laser+vehicle treatment and the untreated control. This demonstrates that C-glycosides can be used to support barrier repair in wounded skin. Thus, these additional immunostaining analysis from the wounded skin model further elucidate the benefits of C-glycosides for wound healing and scar management applications.

### Example 5: Protein Assays

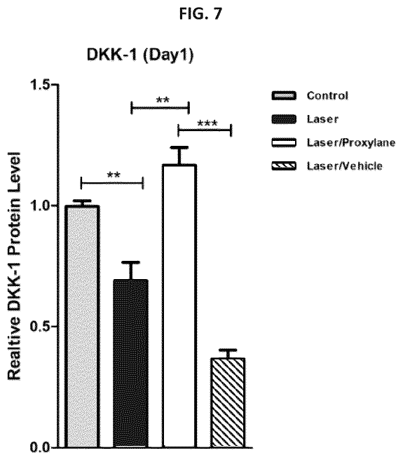

Medium supernatant was collected from the reconstructed skin samples. DKK-1 secretions were measured using a Luminex Assay (R&D Systems) in a panel following the manufacturer's guidelines. Results are provided in FIG. 7. Laser wounded tissue and placebo treated tissue both showed a reduced level of DKK-1 compared to control. In contrast, C-glycoside treatment restored DKK-1 secretion to a level similar to control. Hydroxypropyl tetrahydropyrantriol enhanced expression of DKR-1 compared to laser treatment alone, laser+vehicle treatment and the untreated control. Proxylane appears to modulate the development of acne scars by targeting TGF-β1 the signaling pathways by enhancing the expression of DKK-1 (a key Wnt antagonist molecule that prevents skin fibrosis through TGF beta pathway). This illustrates the ability of C-glycoside to alter the wound environment by increasing the level of DKK-1 and reducing the possibility of developing fibrosis and scarring.

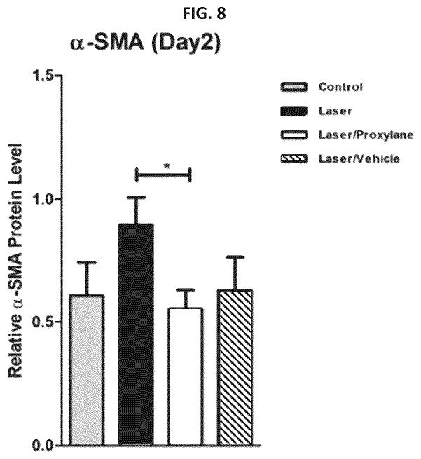

Punched Day 2 normal human tissue cultures tissues were submerged with Tissue Lysis Buffer (R&D Systems) and cryogenically ground 3 times, and further lysed on ice for 30 minutes. Cellular debris was removed by centrifugation at 10,000×g for 10 minutes at 4° C. Aliquots of the supernatant were removed and stored at −80° C. until assayed. α-SMA concentration in ECM was determined using a BCA protein array kit (Absin, abs9232-500) and normalized before ELISA assay (alpha SMA Human ELISA Kit #Abcam, ab240678). Results are provided in FIG. 8. Alpha-smooth muscle actin levels increased in laser wounded reconstructed skin as compared to control. Hydroxypropyl tetrahydropyrantriol (3%) reduced α-SMA expression in a statistically significant manner compared to laser treated tissues that were not further treated. Tissues treated with 3% negative control did not demonstrate such statistically significant improvements. The application of C-glycoside reduced the level of α-smooth muscle actin in laser wounded skin in a statistically significant manner as opposed to laser alone. Such reductions in α-smooth muscle actin were not observed when the placebo was used. The results demonstrate that C-glycoside is beneficial for post procedure wound healing and also for providing a specific mode of action which reduces the potential of scar development by targeting the TGF-β pathway.

Thus, the ELISA analysis from the wounded skin model further elucidate the benefits of C-glycosides for wound healing and scar management applications.

### Example 6: Data Analysis

Each experiment in Examples 2-5 was replicated three times. For immunostaining study, one cryosection per replicate and 4 fluorescence images/section were taken for data analysis. All the data were statistically analyzed by Graph Pad Prism and presented as mean±standard deviation unless otherwise specified. A one-tailed student's t-test was used for data statistical analysis. *, **, and *** denoted significant differences with p value less than 0.01, 0.005 and, 0.001, respectively in FIGS. 4, 6, 7, and 8.

1. A method for improving the appearance of scar tissue, comprising:
(a) treating the scar tissue with at least one skin modification stimulus, and
(b) applying at least one C-glycoside or derivative thereof to the scar tissue.

2. The method of claim 1, wherein the step (b) of applying at least one C-glycoside or derivative thereof to the scar tissue occurs within about 24 hours before and/or after the step (a) of treating the scar tissue with at least one skin modification stimulus.

3. The method of claim 1, wherein the at least one C-glycoside comprises at least one C-xylopyranoside or derivative thereof.

4. The method of claim 1, wherein the at least one C-glycoside is chosen from C13-D-xylopyranoside-n-propan-2-one; Cα-D-xylopyranoside n-propan-2-one; C13-D-xylopyranoside-2-hydroxypropane; Cα-D-xylopyranoside-2-hydroxypropane; 1-(C13-D-fucopyranoside)-propane-2-one; 1-(Cα-D-fucopyranoside)-propan-2-one; 1-(C13-L-fucopyranoside)-propan-2-one; 1-(Cα-L-fucopyranoside)-propane-2-one; 2-one,1-(C13-D-fucopyranoside)-2-hydroxypropane, 1-(Cα-D-fucopyranoside)-2-hydroxypropane; 1-(C13-L-fucopyranoside)-2-hydroxypropane; 1-(Cα-L-fucopyranoside)-2-hydroxypropane; 1-(C13-D-glucopyranosyl)-2-hydroxylpropane; 1-(Cα-D-glucopyranosyl)-2-hydroxylpropane; 1-(C13-D-galactopyranosyl)-2-hydroxylpropane; 1-(Cα-D-galactopyranosyl)-2-hydroxylpropane-1-(C13-D-fucofuranosyl)propan-2-one; 1-(Cα-D-fucofuranosyl)-propan-2-one; 1-(C13-L-fucofuranosyl)-propan-2-one; 1-(Cα-L-fucofuranosyl)-propan-2-one; C13-D-maltopyranoside-n-propan-2-one-Cα-D-maltopyranoside-n-propan-2-one-C13-D-maltopyranoside-2-hydroxypropane; Cα-D-maltopyranoside-2-hydroxypropane; derivatives thereof, or combinations thereof.

5. The method of claim 1, wherein the at least one C-glycoside is chosen from C13-D-xylopyranoside-2-hydroxypropane, Cα-D-xylopyranoside-2-hydroxypropane, or combinations thereof.

6. The method of claim 1, wherein the at least one skin modification stimulus is chosen from one or more of a laser, plasma, radiofrequency, a high-intensity focused ultrasound, an electrical field, heat, a low-level light device, a needle, a microneedle, an abrasive element, or a chemical agent.

7. The method of claim 1, wherein the step of treating the scar tissue with at least one skin modification stimulus comprises treating the scar tissue with one or more of an ablative laser procedure, a non-ablative laser procedure, a microneedling procedure, a high-intensity focused ultrasound procedure, an electroporation procedure, a dermabrasion procedure, a microdermabrasion procedure, a plasma skin rejuvenation procedure, or a chemical exfoliation procedure.

8. The method of claim 1, wherein the step of applying at least one C-glycoside or derivative thereof to the scar tissue comprises:
applying a composition comprising at least one C-glycoside or derivative thereof and at least one carrier to the scar tissue,
wherein the total amount of C-glycosides and derivatives thereof present in the composition ranges from about 0.1% to about 30% by weight, relative to the total weight of the composition.

9. The method of claim 1, comprising, in order:
(a) treating scar tissue with at least one skin modification stimulus to produce modified scar tissue, and
(b) applying at least one C-glycoside or derivative thereof to the modified scar tissue within about 6 hours of step (a).

10. The method of claim 9, wherein the application step (b) is repeated at least one time at least about 12 hours after the first application.

11. The method of claim 10, wherein the application step (b) is repeated at least one time at least about 36 hours after the first application.

12. The method of claim 9, wherein the application step (b) comprises:
applying a composition comprising at least one C-glycoside or derivative thereof and at least one carrier to the scar tissue,
wherein the total amount of C-glycosides and derivatives thereof present in the composition ranges from about 0.1% to about 30% by weight, relative to the total weight of the composition.

13. A method for improving the appearance of scar tissue, comprising:
(a) treating the scar tissue with at least one skin modification stimulus to produce modified scar tissue, and
(b) applying a composition comprising C13-D-xylopyranoside-2-hydroxypropane to the modified scar tissue within about 24 hours after step (a),
wherein the C13-D-xylopyranoside-2-hydroxypropane is present in the composition in an amount ranging from about 0.1% to about 30% by weight, relative to the total weight of the composition.

14. The method of claim 13, wherein the application step (b) is repeated at least one time at least about 12 hours after the first application.

15. The method of claim 14, wherein the application step (b) is repeated at least one time at least about 36 hours after the first application.

16. The method of claim 13, wherein the C13-D-xylopyranoside-2-hydroxypropane is present in the composition in an amount ranging from about 0.5% to about 10% by weight, relative to the total weight of the composition.

17. A method for reducing the formation of scar tissue, comprising:
(a) treating uninjured skin with at least one skin modification stimulus, and
(b) applying at least one C-glycoside or derivative thereof to the uninjured skin.

18. The method of claim 17, wherein the step (b) of applying at least one C-glycoside or derivative thereof to the uninjured skin occurs within about 24 hours after the step (a) of treating the uninjured skin with at least one skin modification stimulus.

19. The method of claim 18, wherein the application step (b) is repeated at least one time at least about 12 hours after the first application following step (a).

20. The method of claim 19, wherein the application step (b) is repeated at least one time at least about 36 hours after the first application following step (a).

21. A method for improving the appearance of modified skin tissue and/or modified scar tissue, comprising applying at least one C-glycoside or derivative thereof to the modified skin tissue and/or modified scar tissue.

22. A kit comprising:
(a) a skin modification stimulus, and
(b) a composition comprising at least one C-glycoside or derivative thereof and at least one carrier,
wherein the total amount of C-glycosides and derivatives thereof present in the composition ranges from about 0.1% to about 30% by weight, relative to the total weight of the composition.

23. A method for reducing the conversion of fibroblasts to myofibroblasts in uninjured skin and/or scar tissue, comprising:
(a) treating uninjured skin and/or scar tissue with at least one skin modification stimulus, and
(b) applying at least one C-glycoside or derivative thereof to the uninjured skin and/or scar tissue.

24. A method for increasing the expression level of DKK-1 in uninjured skin and/or scar tissue, comprising:
(a) treating uninjured skin and/or scar tissue with at least one skin modification stimulus, and
(b) applying at least one C-glycoside or derivative thereof to the uninjured skin and/or scar tissue.

25. A method for increasing the phosphorylation of β-catenin in uninjured skin and/or scar tissue, comprising:
(a) treating uninjured skin and/or scar tissue with at least one skin modification stimulus, and
(b) applying at least one C-glycoside or derivative thereof to the uninjured skin and/or scar tissue.

26. A method for downregulating the signal transduction of TGF-β in uninjured skin and/or scar tissue, comprising:
(a) treating uninjured skin and/or scar tissue with at least one skin modification stimulus, and
(b) applying at least one C-glycoside or derivative thereof to the uninjured skin and/or scar tissue.
